(19) United States
(12) Patent Application Publication
(10) Pub. No.: US 2007/0082943 A1
(43) Pub. Date: Apr. 12, 2007

(54) Process for preparation of substantially pure glimepiride

(76) Inventors: Suresh Mahadev Kadam, Mumbai (IN); Venkatasubramanian Radhakrishnan Tarur, Mumbai (IN); Sanjay Janardham Naik, Mumbai (IN); Sachin Baban Gavhane, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/156,343

(22) Filed: Jun. 17, 2005

(30) Foreign Application Priority Data
Apr. 1, 2005 (IN) 410/MUM/2005

(51) Int. Cl. A61K 31/4015; C07D 207/38

(52) U.S. Cl. 514/423; 548/537

(57) ABSTRACT

The present invention discloses a novel process for purification of trans-4-methyl cyclohexylamine HCl and 4[-2-(3-Ethyl-4-methyl-2-carbonyl pyrrolidine amido) ethyl] benzene sulfonamide used in the synthesis of 3-Ethyl-2,5-dihydro-4-methyl-N-[2-[4-[[[[(trans-4-methyl cyclohexyl)amino]carbonyl]amino]sulfonyl]phenyl]ethyl]-2-oxo-1H-pyrrole-1-carboxamide (I), popularly known as Glimepiride. The present invention also discloses a novel purification of Glimepiride usingS methanolic ammonia and glacial acetic acid to obtain highly pure Glimepiride Form I (I) having the undesired cis isomer below 0.15%. Glimepiride (I) is useful in the treatment of diabetes mellitus.

### RELATED APPLICATION

This application claims priority from Indian patent application No. 410/MUM/2005, filed on 1 Apr. 2005.

### TECHNICAL FIELD

The present invention relates to a process for preparation of substantially pure Glimepiride (Form I). More particularly, the present invention relates to a novel process for purification of trans stereoisomer of 4-methyl cyclohexylamine HCl and of 4-[2-(3-Ethyl-4-methyl-2-carbonyl pyrrolidine amido) ethyl] benzene sulfonamide, key intermediates used in the preparation of 3-Ethyl-2,5-dihydro-4-methyl-N-[2-[4-[[[[(trans-4-methyl cyclohexyl)amino]carbonyl]amino]sulfonyl]phenyl]ethyl]-2-oxo-1H-pyrrole-1-carboxamide commonly known as Glimepiride of Formula I. The invention also relates a novel process for purification of Glimepiride.

### BACKGROUND

Glimepiride, according to U.S. Pat. No. 4,379,785 (EP 031058) issued to Hoechst is prepared via reaction of 4-[2-(3-Ethyl-4-methyl-2-carbonyl pyrrolidine amido) ethyl] benzene sulfonamide (IV) with trans-4-methylcyclohexyl isocyanate (VIII). U.S. Pat. No. 4,379,785 (EP 031058) (hereafter referred to as the '785 patent) discloses heterocyclic substituted sulfonyl ureas, particularly 3-Ethyl-2,5-dihydro-4-methyl-N-[2-[4-[[[[(trans-4-methyl cyclohexyl)amino]carbonyl]amino]sulfonyl]phenyl]ethyl]-2-oxo-1H-pyrrole-1-carboxamide i.e. Glimepiride (I). The '785 patent teaches the preparation of Glimepiride starting from 3-Ethyl-4-methyl-3-pyrolidine-2-one (II) and 2-phenyl ethyl isocyanate to give [2-(3-Ethyl-4-methyl-2-carbonyl pyrrolidine amido) ethyl] benzene (III). The [2-(3-Ethyl-4-methyl-2-carbonyl pyrrolidine amido) ethyl] benzene is converted to the 4-[2-(3-Ethyl-4-methyl-2-carbonyl pyrrolidine amido) ethyl] benzene sulfonamide (IV), by reacting with chlorosulphonic acid, followed by treatment with ammonia solution. This intermediate compound (IV) is then finally reacted with trans-4-methylcyclohexyl isocyanate (VIII) prepared from trans-4-methyl cyclohexylamine HCl (VII) to form Glimepiride.

Glimepiride can also be synthesized by reaction of N-[[4-[2-(3-ethyl-4-methyl-2-oxo-3-pyrroline-1-carboxamido)-ethyl]phenyl]sulphonyl]methylurethane (IX) with trans-4-methyl cyclohexyl amine (VII) as reported by R. Weyer, V. Hitzel in Arzneimittel Forsch 38, 1079 (1988).

trans-4Methylcyclohexyl isocyanate (VIII) is prepared from trans-4-methyl cyclohexyl amine HCl (VII), by phosgenation.

H. Ueda et. al., S.T.P Pharma Sciences, 13(4) 281-286, 2003 discloses a novel polymorph of Glimepiride, Form II obtained by recrystallisation from a solvent mixture of ethanol and water. It also discloses that earlier known form is Form I. Reported solvents for obtaining Form I are methanol, acetonitrile, chloroform, butyl acetate, benzene and toluene.

An alternative route is disclosed in WO03057131(Sun Pharmaceutical), where 3-ethyl-4-methyl-2,5-dihydro-N-(4-nitrophenyloxycarbonyl)-pyrrole-2-one is treated with 4-(2-aminoethyl)-benzene sulphonamide to obtain 4-[2-(3-Ethyl-4-methyl-2-carbonyl pyrrolidine amido) ethyl] benzene sulfonamide (IV) which was then converted to Glimepiride (I). However, nonavailability of raw material and the yield being poor, the process as described in U.S. Pat. No. 4,379,785 is preferred.

To obtain Glimepiride of highest purity, following intermediates should be of highest quality:

a) 4-[2-(3-Ethyl-4-methyl-2-carbonyl pyrrolidine amido) ethyl] benzene sulfonamide (IV) with lowest possible content of ortho and meta isomers.

b) Trans-4-methyl cyclohexyl amine (VII) and its respective isocyanate (VIII) should have lowest content of the cis isomer.

The preparation of the 4-[2-(3-Ethyl-4-methyl-2-carbonyl pyrrolidine amido) ethyl] benzene sulfonamide is well disclosed in the patent U.S. Pat. No. 4,379,785. It is prepared by condensation of 3-ethyl-4-methyl-3-pyrrolidine-2-one of Formula (II) with 2-phenyl ethyl isocyanate. The condensed product is then chlorosulphonated with chlorosulphonic acid followed by ammonolysis with liq. ammonia to give compound of Formula (IV). The purity is not well documented in the patents, and by following the patented process, ˜85 to 88% of desired para isomer is obtained. This is evident as the chlorosulphonation is ortho-para directing.

Hence, there is a need to develop purification process to maintain undesired ortho and meta isomers below 0.1%.

The other key intermediate trans-4-methylcyclohexyl amine HCl (VII) should preferably have lowest possible content of the cis isomer. The commonly used procedure is reduction of 4-methyl cyclohexanone oxime (V) with sodium in alcohol, preferably ethanol.

T. P. Johnston, et. al., J. Med. Chem., 14, 600-614 (1971); H. Booth, et. al., J. Chem. Soc (B) 1971, 1047-1050 and K. Ramalingam et. al., Indian Journal of Chem Vol. 40, 366-369 (April 1972) all report the abovementioned reduction. The amine obtained via this process typically contains between 8 to 10% of the cis isomer. However, use of high excess sodium metal (25 eqv.) for reduction makes process commercially and environmentally unviable. Also, the purification of trans amine from the mixture via the distillation is very difficult as the boiling points differ only by about 2° C. Also there is an inherent drawback of said free amine as, it immediately forms carbonate salt. Further purification of the amine to reduce the cis content via crystallization of its salt is not sufficiently documented. Prior art describes purification of crude trans-4-methylcyclohexylamine HCl by crystallization of its hydrochloride but the yield and purity are not sufficiently discussed. A description of such purification is provided in J. Med. Chem, 14, 600-614 (1971), wherein trans-4-methylcyclohexylamine HCl is obtained by triple crystallization in acetonitrile of the crude hydrochloride (m.p. 260° C.) in 27% yield.

WO 2004073585 (Zentiva) describes a process for preparation of trans-4-methylcyclohexylamine HCl wherein the highlights of the invention are the use of sodium metal and purification via the pivalic acid salt. However drawbacks of the process are use of sodium metal, which is hazardous and pivalic acid which is expensive. The overall yield is ˜40%.

Thus considering the current stringent pharmacopieal requirements for cis content, there is a need for obtaining Glimepiride having cis impurity content well below 0.15% by a cost effective process.

Key factors in the production of Glimepiride are:

a) Substantial purity of trans-4-methyl cyclohexyl amine HCl (VII) with the lowest possible content of the cis isomer.

b) Substantial purity of 4-[2-(3-Ethyl-4-methyl-2-carbonyl pyrrolidine amido) ethyl] benzene sulfonamide (IV) with the lowest possible content of the ortho and meta isomer.

The purity of intermediate compound of Formula (IV) when prepared by the process disclosed in '785 patent, was found to be 82 to 85% by HPLC.

### OBJECTS OF THE INVENTION

The object of the present invention is to prepare trans-4-methyl cyclohexylamine HCl of Formula (VII), a key intermediate with a substantially high content of the trans-isomer.

2) Another object of the present invention is to prepare 4-[2-(3-Ethyl-4-methyl-2-carbonyl pyrrolidine amido) ethyl] benzene sulfonamide of Formula (IV) of higher purity.

3) Yet another object of the present invention is to prepare Glimepiride of Formula (I) of pharmaceutically acceptable quality by employing the intermediate compound viz., 4-[2-(3-Ethyl-4-methyl-2-carbonyl pyrrolidine amido) ethyl] benzene sulfonamide of Formula (IV).

4) A further object of the present invention is to purify Glimepiride to get pharmaceutically acceptable quality (i.e. meta and ortho isomers content below 0.1%) using methanolic ammonia to obtain polymorph Form I of Glimepiride.

### SUMMARY

The present invention discloses a process for

a) Purification of intermediate compound of Formula (IV) viz. 4-[2-(3-Ethyl-4-methyl-2-carbonyl pyrrolidine amido) ethyl] benzene sulfonamide using a mixture of methanol and acetone.

b) Purification of intermediate compound of Formula (VII) viz. trans-4-methyl cyclohexylamine HCl using methanol, acetone and toluene or mixtures thereof.

c) Reacting a compound of formula 4-[2-(3-Ethyl-4-methyl-2-carbonyl pyrrolidine amido) ethyl] benzene sulfonamide of Formula (IV) with trans-4-methyl cyclohexyl isocyanate of Formula (VIII) to obtain the compound of Formula (I).

d) Purification of Glimepiride (I) with methanolic ammonia and glacial acetic acid to obtain Glimepiride Form I in substantially pure form.

These and other aspects of the present invention will now be described in more detail further herein.

### DESCRIPTION OF DRAWING

FIG. 1 show the XPRD of Glimeperide obtained according to the example 5.

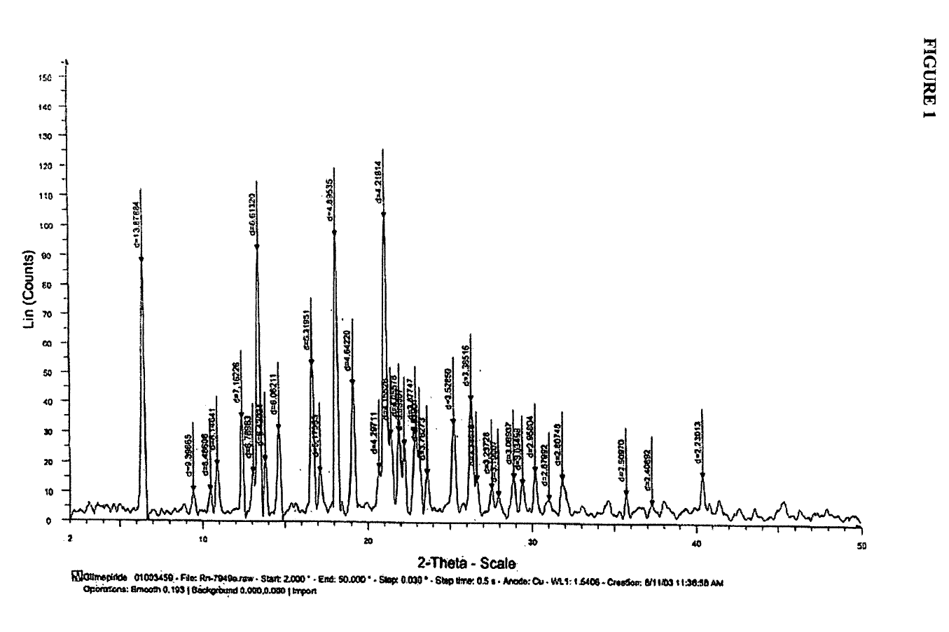

### DETAILED DESCRIPTION

The present invention provides a novel process for the purification of
a) trans-4-methyl cyclohexylamine hydrochloride (VII).
b) 4-[2-(3-Ethyl-4-methyl-2-carbonyl pyrrolidine amido) ethyl] benzene sulfonamide (IV)
c) Glimepiride (I)

The present invention relates to a purification process to obtain Glimepiride (I) in a highly pure form. However, the inventive process can be used to prepare any compound within the scope of compound (I) as shown in schemes I to III.

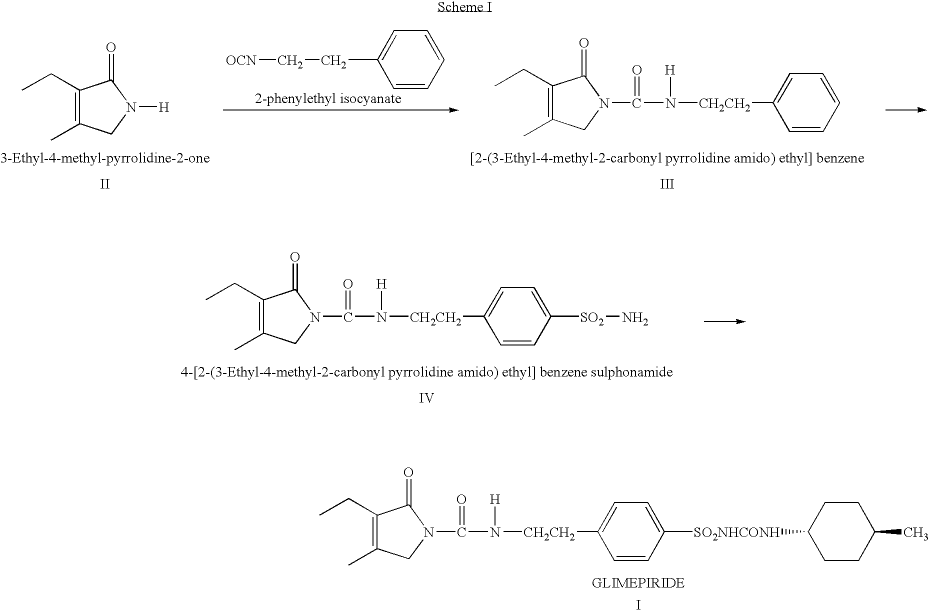

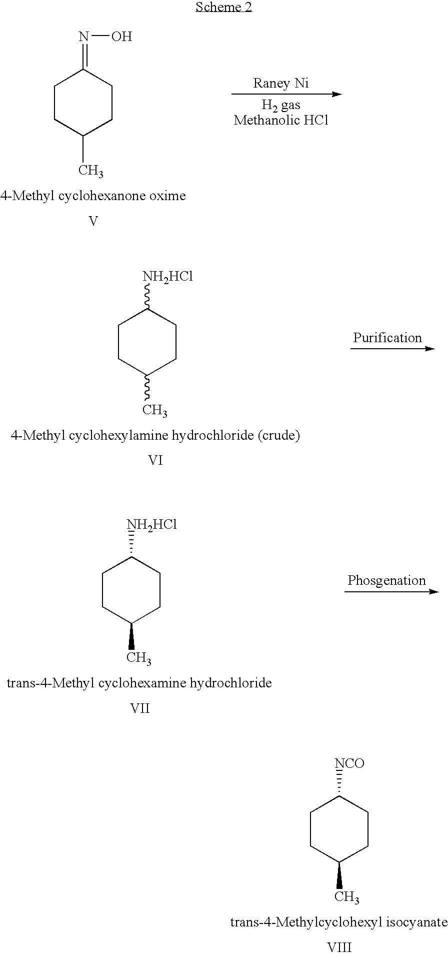

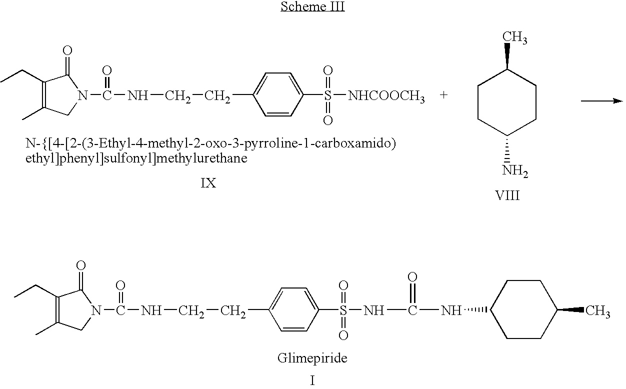

The purification of trans-4-methyl cyclohexylamine HCl (VII) is accomplished by using an appropriate solvent combination. The mixture of cis/ trans stereoisomers (i.e. 50:50) were dissolved in diluted methanol and the desired trans isomer is coprecipitated by adding acetone to it. The process is repeated with different proportions of the solvent mixture to get the trans-4-Methyl cyclohexylamine HCl (VII) >99.5% with cis isomer less than 0.15%. The overall yield from 4-methyl cyclohexanone is ˜30%. The purification has been achieved using a solvent mixture of alcohol and ketone. A preferred alcohol for dissolution is an aliphatic one wherein carbon chain may be preferably C1-C4. Preferably methanol is used to dissolve the crude trans-4-Methyl cyclohexylamine HCl. The ratio of substrate:methanol:acetone is fixed at 1:1.5:6 for achieving the desired purity. The cosolvent used for precipitation is an aliphatic ketone. The preferred ketone is acetone. The precipitation is carried out at a temperature between 20 to 50° C., preferably between 30 to 50° C. and most preferably at about 40° C. The addition of acetone is carried out over a period of 2 to 6 hrs, more preferably for about 2 to 4 hrs and most preferably in about 3 hrs. The compound thus obtained has a purity >95% by gas chromatography.

The enriched trans-4-Methyl cyclohexylamine HCl (VII) (>95%) is further purified using different proportions of the same solvent mixture. The enriched trans isomer is dissolved in alcohol and reprecipitated using an aliphatic ketone. The ratio of substrate:methanol:acetone ratio is fixed at 1:1.5:13.6 for obtaining purity greater than 99.8%.

Preferred alcohol is aliphatic wherein the carbon chain may be preferably C1-C4. Preferably methanol is used to dissolve the enriched trans-4-Methyl cyclohexylamine HCl (VII). The cosolvent used for precipitation is an aliphatic ketone. The preferred ketone is acetone. The precipitation is preferably carried out at a temperature between 20 to 50° C., more preferably between 30 to 50° C. The addition of acetone is preferably carried out over a period of 2 to 6 hrs, more preferably for about 2 to 4 hrs. The purity obtained is greater than 99.8% by gas chromatography. The cis content is controlled well below 0.15%. Yield obtained is 70%.

The purity of other key intermediate i.e. 4-[2-(3-Ethyl-4-methyl-2-carbonyl pyrrolidine amido) ethyl] benzene sulfonamide (IV) is also not well documented in the literature. U.S. Pat. No. 4,379,785 (EP 031058) reports condensation of crude 4-[2-(3-Ethyl-4-methyl-2-carbonyl pyrrolidine amido) ethyl] benzene sulfonamide (IV) with trans-4-methyl cyclohexyl isocyanate (VIII) to obtain Glimeperide (I). However, using this crude sulfonamide there is always a possibility of getting undesired ortho and meta isomers in Glimepiride. The present invention relates to a purification process of 4-[2-(3-Ethyl-4-methyl-2-carbonyl pyrrolidine amido) ethyl] benzene sulfonamide of Formula (IV) with a mixture of hydrocarbon and alcohol. The hydrocarbon can be aliphatic, alicyclic or aromatic. The hydrocarbon is further selected from hexane, heptane ,cyclohexane, toluene or mixtures thereof preferably, toluene. The alcohol used for crystallization is an aliphatic one, wherein, the carbon chain may be preferably C1 to C4. Preferably methanol is used with toluene for recrystallisation. The desired para isomer, having a purity greater than 95% is obtained. The undesired ortho isomer reduces from 8-10% to 1-2%. Repeated crystallization using alcohol/ketone combination minimizes the ortho and meta isomers well below 0.5%, preferably 0.2%. The alcohol used in this combination is an aliphatic alcohol, preferably methanol while the ketone used is an aliphatic ketone, preferably acetone in the volume ratio of 2:8, preferably 4:6. The purity of the desired para isomer thus obtained is greater than 99% by HPLC.

The said condensation of 4-[2-(3-Ethyl-4-methyl-2-carbonyl pyrrolidine amido) ethyl] benzene sulfonamide (IV) to Glimepiride (I) is well document U.S. Pat. No. 4,379,785 (EP 031058). However, content of other isomer i.e. ortho and meta isomers of Glimepiride is not reported and hence there is a need to have a purification process to control these isomers below 0.1%.

This invention further describes purification of crude Glimepiride. The purification has been reported by crystallization from an appropriate solvent selected from dioxane, THF, dimethoxyethane, dimethoxymethane, acetic acid, DMSO, acetone, acetonitrile, DMF or mixtures thereof. However due to high polarity of the Glimepiride large volumes of solvents were required for crystallization.

Hence novel purification methodology using methanolic ammonia has been established to minimize the isomeric impurities as well as degradation of Glimepiride at higher temperatures.

The purification of Glimepiride is carried out in alcoholic ammonia, preferably in aliphatic alcohol having C1-C4 carbon chain. Preferably 6 volumes of methanol is used for purification. Dry ammonia gas is purged at a temperature of 10 to 30° C. for dissolution, preferably, at 15 to 25° C. and most preferably at 20° C. The reprecipitation of the product is done by neutralizing the ammonium salt of Glimepiride with acid selected from sulphuric acid, hydrochloric acid and acetic acid; preferably acetic acid at pH 5.5 to 6.0. The reprecipitation is preferably done at 15 to 20° C. The product thus obtained by this process is consistently found to be Form I.

The XRPD, IR, DSC matches values reported by H. Ueda et. al., S.T.P Pharma Sciences, 13(4), 281-286, 2003 as presented in Table 1.

TABLE 1
XRPD peaks of Glimepiride

| Angle 2-Theta° | d value Angstrom | Intensity % |
|---|---|---|
| 6.363 | 13.87884 | 84.6 |
| 9.404 | 9.39665 | 9.3 |
| 10.416 | 8.48606 | 9.5 |
| 10.852 | 8.14641 | 17.9 |
| 12.348 | 7.16226 | 33.1 |
| 13.029 | 6.78963 | 15.5 |
| 13.378 | 6.61320 | 89.0 |
| 13.760 | 6.43024 | 19.4 |
| 14.600 | 6.06211 | 29.5 |
| 16.652 | 5.31951 | 51.0 |
| 17.118 | 5.17583 | 15.9 |
| 18.107 | 4.89535 | 93.9 |
| 19.103 | 4.64220 | 44.2 |
| 20.653 | 4.29711 | 17.1 |
| 21.044 | 4.21814 | 100.0 |
| 21.367 | 4.15526 | 28.1 |
| 21.897 | 4.05578 | 29.2 |
| 22.207 | 3.99987 | 24.9 |
| 22.917 | 3.87747 | 28.3 |
| 23.150 | 3.83901 | 21.5 |
| 23.626 | 3.76273 | 15.4 |
| 25.219 | 3.52859 | 31.8 |
| 26.306 | 3.38516 | 39.5 |
| 26.618 | 3.34618 | 13.6 |
| 27.531 | 3.23728 | 11.0 |
| 27.946 | 3.19007 | 8.4 |
| 28.877 | 3.08937 | 14.3 |
| 29.406 | 3.03498 | 12.4 |
| 30.189 | 2.95804 | 16.4 |
| 31.028 | 2.87992 | 7.2 |
| 31.849 | 2.80748 | 14.0 |
| 35.749 | 2.50970 | 9.0 |
| 37.298 | 2.40892 | 5.9 |
| 40.413 | 2.23013 | 15.1 |

The formation of Form I is further confirmed by the IR and DSC data. Bands at 3290 cm⁻¹ & 3370 cm⁻¹ confirm Form I.

DSC shows only one endotherm peak corresponding to its melting point together with its decomposition at 207.7 C

The present invention comprises,

a) Purification of trans-4-methyl cyclohexylamine HCl (VII);

b) Purification of 4-[2-(3-Ethyl-4-methyl-2-carbonyl pyrrolidine amido) ethyl] benzene sulfonamide (IV); and

c) Purification of Glimepiride.

Thus the present invention provides exceptionally pure Glimepiride with a content of the undesirable cis isomer lower than 0.15%

The present invention is illustrated in further detail with reference to the following non-limiting examples:

### EXAMPLE 1

### trans-4-Methyl cyclohexylamine HCl (VII)

1.5 Kg of crude 4-Methyl cyclohexyloxime (V) was dissolved in 8.33 L Methanol. To this 0.15 Kg Raney nickel was added. Then the mixture was hydrogenated at 4-5 Kg/cm² pressure at 50 to 55° C. After the absorption of H₂ ceases, the reaction mass is cooled down and filtered. From resulting reaction mixture, methanol was distilled completely. Crude concentrated oil obtained is cooled to 15 to 20° C. to which methanolic hydrochloric acid (12 to 13%) is added slowly, when the product i.e. 4-Methylcyclohexylamine HCl precipitates out. The yield obtained 1.5 Kg of crude 4-methyl cyclohexylamine HCl (85%) with ˜50% content of trans isomer. The crude 4-Methyl cyclohexylamine HCl 1.5 Kg (wet) was further purified in methanol/acetone mixture. The crude 4-methyl cyclohexylamine HCl (1.5 Kg) was dissolved in 2.25 L of methanol at 25 to 30° C. Slowly started addition of 13.5 L of acetone over a period of 3 hrs. The trans-4-methyl cyclohexylamine HCl precipitated out. Yield 0.6 Kg. The purity achieved of trans isomer is >95%. The cis isomer at this stage is ˜2 to 3%.

The trans-4-methyl cyclohexylamine HCl (0.6 Kg) thus obtained is again taken in 0.9 L of methanol and is dissolved completely at 25 to 30° C. 8.1 L acetone is added slowly over a period of 3 hrs when pure trans isomer precipitates out completely. The purity achieved at this stage is >99.8% and cis isomer well below 0.15%. The yield thus obtained after the second purification is 0.48 Kg of trans-4-Methyl cyclohexylamine HCl (27.2% yield calculated on the starting oxime). Purity of the desired trans isomer is greater than 99.8% by G.C.

Melting point of the trans-4-methyl cyclohexylamine HCl thus obtained is 262° C. to 263° C.

### EXAMPLE 2

### Preparation of 4-[2-(3-Ethyl-4-methyl-2-carbonyl pyrrolidine amido) ethyl] benzene sulfonamide (IV)

3-Ethyl-4-methyl-2,5-dihydro-1H-pyrrole-2-one (II) (1.0 Kg) and β-phenylethyl isocyanate (1.488 Kg) were mixed in anhydrous toluene (4.0 L) and refluxed for 4 hrs. The toluene was distilled off and hexane (8.0 L) was added to the reaction mixture at 50° C. The product precipitated is cooled to 0 to 5° C. to obtain the solid compound viz. 4-[2-(3-Ethyl-4-methyl-2-carbonyl pyrrolidine amido) ethyl] benzene (2.17 Kg). It was filtered & washed with 2.0 L of hexane.

To a cooled (15 to 25° C.) solution of chlorosulfonic acid (2.8 L), 4-[2-(3-Ethyl-4-methyl-2-carbonyl pyrrolidine amido)ethyl] benzene (2.0 Kg) was added in small portions over a period of 2 to 3 hrs. Further it was stirred for 30 min at this temperature and then temperature was gradually raised to 30 to 35° C. The reaction mass is stirred further for 2 hrs. The reaction mixture was then quenched into ice-water and stirred for 1 hr and filtered to obtain the product 4-[2-(3-Ethyl-4-methyl-2-carbonyl pyrrolidine amido)ethyl] benzene sulfonyl chloride (2.0 kg). To a cooled (15 to 20° C.) solution of diluted ammonia (1.4 L) 4-[2-(3-Ethyl-4-methyl-2-carbonyl pyrrolidine amido) ethyl] benzene sulfonyl chloride was added in small portion over 1 to 2 hrs. The reaction mixture was then heated to 70° C. for 2 hrs when ammonolysis is complete. The product converted is then stirred for 1 hr at R.T. and filtered and dried at 90 to 100° C. to obtain crude 4-[2-(3-Ethyl-4-methyl-2-carbonyl pyrrolidine amido) ethyl] benzene sulfonamide (2.2 Kg) having HPLC purity in the range of 82 to 88%. The crude compound 4-[2-(3-Ethyl-4-methyl-2-carbonyl pyrrolidine amido) ethyl] benzene sulfonamide (2.2 Kg) is then purified from mixture of organic solvents chosen from Methanol, Acetone & toluene.

### EXAMPLE 3A

### Purification of 4-[2-(3-Ethyl-4-methyl-2-carbonyl pyrrolidine amido)ethyl] benzene sulfonamide (IV)

1^{st} Purification

In a reaction vessel containing Toluene (12.0 L), 4-[2-(3-Ethyl-4-methyl-2-carbonyl pyrrolidine amido) ethyl] benzene sulfonamide (2.0 Kg) was charged at 25 to 30° C. Slowly the temperature was raised to 60 to 65° C. and methanol (5.0 L) was added via the dosing tank slowly when the product dissolved completely. Refluxed it for 0.5 hr. Charcoalised and filtered the product in another reaction vessel. Distill off toluene/methanol mixture till total recovery about 65% under vacuum. White crystalline product precipitated out. After the recovery, cool the reaction mass to 15 to 20° C. The resulting crystallized solid product was filtered and washed two times with chilled acetone (about 2 L) each. The resulting product was dried at 90 to 100° C. in air oven till constant weight to obtain about 1.4 Kg of 4-[2-(3-Ethyl-4-methyl-2-carbonyl pyrrolidine amido)ethyl] benzene sulfonamide with greater than 95% HPLC purity.

### EXAMPLE 3B

### Purification of 4-[2-(3-Ethyl-4-methyl-2-carbonyl pyrrolidine amido)ethyl] benzene sulfonamide (IV)

2^{nd} Purification

In a reaction vessel containing Acetone (8.4 L), (1.4 Kg) of 1st purified 4-[2-(3-Ethyl-4-methyl-2-carbonyl pyrrolidine amido)ethyl] benzene sulfonamide was charged at 25 to 30° C. slowly and the temperature was raised to 55 to 60° C. Methanol was added (5.6 L) via the dose tank at this reflux temperature to dissolve it completely. Refluxed it for further 30 min. Distilled off acetone/ methanol mixture till total recovery about 65 to 70%. White crystalline product precipitated out. After the recovery slowly cooled the product to 15 to 20° C. The resultant solid product was filtered, washed two times with chilled acetone (1.4 L) each. The 4-[2-(3-Ethyl-4-methyl-2-carbonyl pyrrolidine amido) ethyl] benzene sulfonamide was dried at 90 to 100° C. in air oven till constant weight to obtain about 1.12 Kg of 4-[2-(3-Ethyl-4-methyl-2-carbonyl pyrrolidine amido)ethyl] benzene sulfonamide (IV) with greater than 99.5% purity with other isomers i.e. ortho and meta well below 0.2% respectively.

### EXAMPLE 4

### Preparation of 3-Ethyl-2,5-dihydro-4-methyl-N-[2-[4-[[[[(trans-4-methyl cyclohexyl)amino]carbonyl]amino]sulfonyl]phenyl]ethyl]-2-oxo-1 H-pyrrole-1-carboxamide (I).

In a reaction vessel containing (24.2 L) Acetone, 4-[2-(3-Ethyl-4-methyl-2-carbonyl pyrrolidine amido)ethyl] benzene sulfonamide (1.0 Kg) and potassium carbonate (0.46 Kg) was added and refluxed at about 55 to 60° C. for 1 hr. trans-4-Methyl-cyclohexyl isocyanate was obtained by method known in art from trans-4-methyl-cyclohexylamine. A solution of trans-4-methyl-cyclohexyl isocyanate (0.515 Kg) in toluene (5 L) was prepared and added to the above reaction mixture. This reaction mixture is refluxed for 12 hrs, then cooled. To this cooled reaction mass charge 27 L of water. The reaction mass was filtered and the pH was adjusted to 5.5 to 6.0 by adding acetic acid at about 20 to 25° C. The solid obtained was filtered and washed with water. The 3-Ethyl-2,5-dihydro-4-methyl-N-[2-[4-[[[[(trans-4-methyl cyclohexyl)amino]carbonyl]amino]sulfonyl]phenyl]ethyl]-2-oxo-1H-pyrrole-1-carboxamide (I) obtained is then dried at 90 to 100° C. till constant weight. Yield of the product is 86.3%.

### EXAMPLE 5

### Purification of 3-Ethyl-2,5-dihydro-4-methyl-N-[2-[4-[[[[(trans-4-methyl cyclohexyl)amino]carbonyl]amino]sulfonyl]phenyl]ethyl]-2-oxo-1H-pyrrole-1-carboxamide (I)

In a reaction vessel containing 6.0 L methanol and 1.0 Kg crude Glimepiride, dry ammonia gas was purged at 20 to 25° C. till all Glimepiride dissolves and a clear solution is obtained. This homogeneous mass was then charcoalised, filtered and finally neutralized with Glacial acetic acid to pH 5.5 to 6.0, till the entire product precipitates out. The pure Glimepiride was then filtered and dried at 65° C. to 70° C. till constant weight. Yield obtained was ˜90%.

1) A process for the preparation of substantially pure 3-Ethyl-2,5-dihydro-4-methyl-N-[2-[4-[[[[(trans-4-methyl cyclohexyl)amino]carbonyl]amino]sulfonyl]phenyl]ethyl]-2-oxo-1H-pyrrole-1-1carboxamide (Glimepiride) Form I of formula (I) wherein, said process comprises, 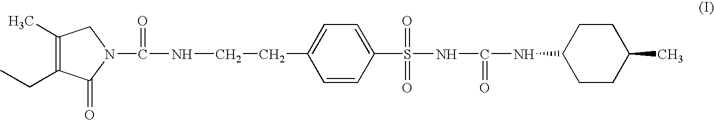
a) Purifying 4-[2-(3-Ethyl-4-methyl-2-carbonyl pyrrolidine amido)ethyl] benzene sulfonamide of Formula (IV) with a mixture of solvents using a hydrocarbon, alcohol and a ketone;
b) Purifying trans-4-methyl cyclohexylamine HCl (VII) with a mixture of alcohol and ketone;
c) Converting trans-4-methyl cyclohexylamine HCl (VII) to trans-4-methyl cyclohexylamine isocyanate (VIII) by a method known in the art;
d) Condensing 4-[2-(3-Ethyl-4-methyl-2-carbonyl pyrrolidine amido)ethyl] benzene sulfonamide (IV) with trans-4-methyl cyclohexylamine isocyanate (VIII) to obtain Glimepiride by a method known in the art and
e) purifying the Glimepiride to obtain substantially pure Glimeperide in polymorphic form I.

2) The process as claimed in claim 1, wherein said purification of 4-[2-(3-Ethyl-4-methyl-2-carbonyl pyrrolidine amido)ethyl] benzene sulfonamide (IV) 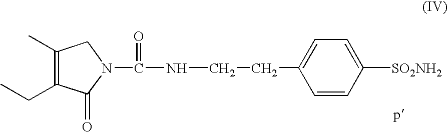
is carried out by crystallizing from a mixture of hydrocarbon and alcohol and further recrystallizing from a mixture of ketone and alcohol.

3) The process as claimed in claim 1 and claim 2 wherein hydrocarbon is selected from the group including aliphatic, alicyclic and aromatic hydrocarbons; preferably selected from hexane, heptane, cyclohexane and toluene or a mixture thereof.

4) The process as claimed in claim 1 and 2 wherein alcohol is selected from the group of C1 to C4 aliphatic alcohols preferably methanol.

5) The process according to any of the preceding claims wherein 4-[2-(3-Ethyl-4-methyl-2-carbonyl pyrrolidine amido)ethyl] benzene sulfonamide (IV) is recrystallised from mixture of toluene and methanol in the volume ratio of 6:2.5.

6) The process as claimed in claim 5 wherein purity of 4-[2-(3-Ethyl-4-methyl-2-carbonyl pyrrolidine amido)ethyl] benzene sulfonamide (IV) obtained is 95%.

7) The process as claimed in claim 2, wherein 4-[2-(3-Ethyl-4-methyl-2-carbonyl pyrrolidine amido)ethyl] benzene sulfonamide (IV) is further recrystallised from a mixture of ketone and alcohol.

8) The process as claimed in claim 7 wherein the ketone is selected from the group aliphatic ketones, preferably acetone; and the alcohol is selected from the group of C1 to C4 aliphatic alcohol, more preferably methanol.

9) The process as claimed in claim 7 to 8 wherein 4-[2-(3-Ethyl-4-methyl-2-carbonyl pyrrolidine amido) ethyl] benzene sulfonamide (IV) is recrystallised using a mixture of acetone and methanol in the volume ratio of 6:4.

10) The process as claimed in claim 7 wherein purity of 4-[2-(3-Ethyl-4-methyl-2-carbonyl pyrrolidine amido) ethyl] benzene sulfonamide (IV) is greater than 99.2% with both ortho and meta isomer below 0.2%.

11) The process as claimed in claim 1 wherein trans-4-methyl cyclohexylamine HCl (VII) 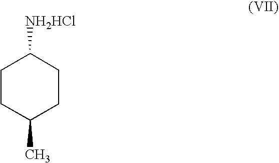
is recrystallised from a mixture of solvent, selected from the group of C1 to C4 alcohols and a ketone as a solvent selected from the group of aliphatic ketones.

12) The process as claimed in claim 1 and claim 11 wherein trans-4-methyl cyclo hexylamine HCl (VII) is recrystallised from a mixture of methanol and acetone in the volume ratio of 1.5:6 to obtain purity greater than 95%.

13) The process as claimed in claim 12 wherein trans-4-methyl cyclo hexylamine HCl (VII) is recrystallised further from solvent mixture of methanol and acetone in the volume ratio of 1.5:13.6 to obtain purity of 99.8%.

14) The process as Claimed in claim 1 wherein said purification of Glimiperide comprises dissolving 3-Ethyl-2,5-dihydro-4-methyl-N-[2-[4-[[[[(trans-4-methyl clohexyl)amino]carbonyl] amino]sulfonyl]phenyl]ethyl]-2-oxo-1H-pyrrole-1-carboxamide (Glimepiride) compound (I) in an alcohol; using a base; optionally charcoaling the resultant clear solution; adjusting the pH preferably to 5.5 to 6.0 using an acid and isolating the pure Glimepiride.

15) The process as Claimed in claim 1 and claim 14 wherein base is preferably Iiammonia and the alcohol is selected from the group of C1 up to C4 alcohol, preferably methanol.

16) The process as claimed in claim 1 and claim 14 wherein acid is selected from the group of hydrochloric acid, sulphuric acid or acetic acid, preferably acetic acid.

17) Glimepiride according to any of the preceding claims 14 to 16 has purity greater than 99.8%.

18) A process for the preparation of substantially pure Glimepiride as substantially described and exemplified herein with reference to the foregoing examples 1 to 5.
